(19) United States
(12) Patent Application Publication
(10) Pub. No.: US 2025/0121215 A1
(43) Pub. Date: Apr. 17, 2025

(54) TFUS SYSTEM CONFIGURED WITH SIMPLIFIED PROBES

(71) Applicant: Sanmai Technologies, PBC, Sunnyvale, CA (US)

(72) Inventor: Christopher DAFT, Tucson, AZ (US)

(21) Appl. No.: 18/909,120

(22) Filed: Oct. 8, 2024

Related U.S. Application Data
(60) Provisional application No. 63/589,928, filed on Oct. 12, 2023.

(51) Int. Cl. A61N 7/00

(52) U.S. Cl. CPC A61N 7/00; A61N 2007/0026; A61N 2007/0091

(57) ABSTRACT

A transcranial Focused Ultrasound (tFUS) system employs a probe array for steering and focusing a neuromodulation beam, wherein the probe array comprises an array of piezo-electric elements and the size of each element is larger in elevation than in azimuth. A “fuel gauge” user interface is employed to assist in selecting optimal probe alignments. Guidance information is enhanced using optical data provided by a mobile device incorporating a camera or a LIDAR device. A simulated prediction of the ultrasound neuromodulation beam is overlayed on an image of the brain.

### CROSS-REFERENCE TO RELATED APPLICATIONS

This application claims the priority benefit of U.S. Provisional Patent Application No. 63/589,928, filed Oct. 12, 2023 and entitled “TFUS SYSTEM CONFIGURED WITH SIMPLIFIED PROBES”. The subject matter of this related application is hereby incorporated herein by reference.

### BACKGROUND

U.S. Pat. No. 10,792,519, FOCUSED ULTRASOUND PROBE NAVIGATION SYSTEM, issued to Wurster et al, describes a focused ultrasonic probe navigation system in the prior art. A housing contains an ultrasonic device, and an alignment system moves the housing and the ultrasonic device within the housing, to align a focused point of ultrasonic energy aligned in three dimensions with a target location in a subject. In repeated therapy sessions alignment is re-established without requiring an MRI system. This system is further described in the Detailed Description.

U.S. Pat. No. 6,471,651, LOW POWER PORTABLE ULTRASONIC DIAGNOSTIC EQUIPMENT, issued to Hwang et al, describes a portable ultrasonic diagnostic equipment in the prior art. The system includes an array probe, a beamformer, signal processing and imaging circuitry, and a display for depicting echoed signals. This system is further described in the detailed description.

Young Goo Kim et al published a paper in the Journal of Clinical Medicine, “NEUROMODULATION USING TRANSCRANIAL FOCUSED ULTRASOUND ON THE BILATERAL MEDIAL PREFRONTAL CORTEX”, June 2022. This system is further described in the Detailed Description.

### BRIEF DESCRIPTION OF THE DRAWINGS

FIG. 1 illustrates a prior art tFUS system including a three-dimensional alignment system.

FIG. 2 illustrates a prior art tFUS system including a beamformer to focus transmitted and received echoes.

FIG. 3 illustrates a low-intensity tFUS system using neuronavigation presented in a paper by Young Goo Kim et al.

FIG. 4 illustrates a “1.75 D” array wherein slice patterns are larger in elevation than in azimuth, in an embodiment of the present disclosure.

FIG. 5 illustrates a tFUS system incorporating a “fuel gauge” for indicating when the best slice is selected for the acquisition plane, in embodiments of the present disclosure.

FIG. 6 illustrates a tFUS system wherein the 1.75 D probe array is rotatable for achieving an optimal orientation of the acquisition plane, in an embodiment of the present disclosure.

FIG. 7 illustrates a tFUS system wherein a mobile imaging device is deployed in cooperation with an optical registration feature, in an embodiment of the present disclosure.

FIG. 8 illustrates a block diagram of a tFUS system wherein a simulated ultrasound beam pattern is overlayed on brain anatomy produced by MRI, in an embodiment of the present disclosure.

### DETAILED DESCRIPTION OF THE INVENTION

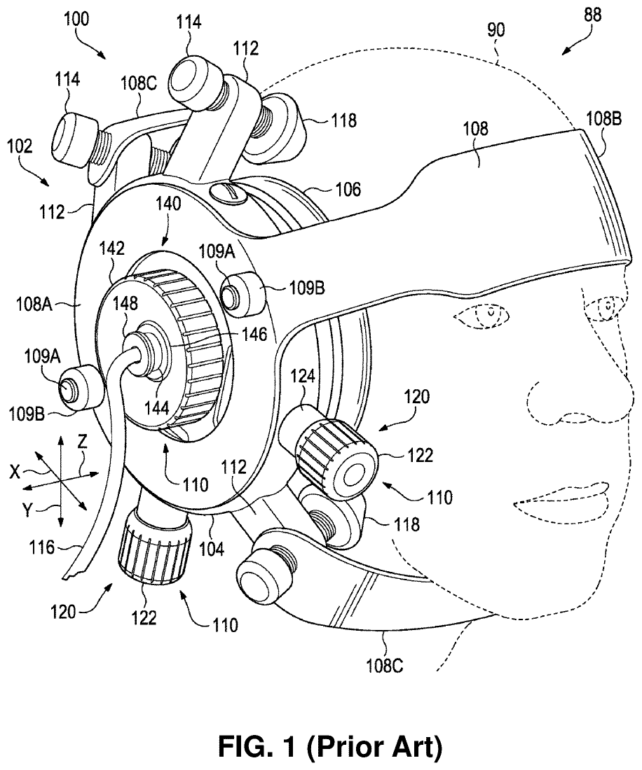

FIG. 1, prior art, shows a perspective view of a focused ultrasonic Probe Navigation System (TNS) 100 described in U.S. Pat. No. 10,792,519. TNS 100 may be attached to a patient 88 and may apply ultrasonic energy to a target location within head 90 of patient 88. However, TNS 100 may apply any type of sonic, magnetic, or any other alternative energy to any target location within any body part of patient 88. TNS 100 may be used on human patients or animal patients. A housing assembly 102 comprises an outer housing 104 attached to a movable inner housing 106. A probe may be located within inner housing 106. A power cable 116 may attach to the probe and extend up through the inner housing 106 and outer housing 104. A first vertical strap 108C attaches to elevating screws 114 and wraps around the top of head 90 and underneath the chin of patient 88. A second horizontal strap 108 includes a ring-shaped section 108A that attaches to an outside surface of outer housing 104 via screws 109A and nuts 109B and a headband section 108B that wraps around the front over the eyes and back of head 90. While shown attached to head 90 straps 108 or other attachment devices, may attach housing assembly 102 to other body parts of patient 88. The housing assembly 102 may be attached by straps 108 to the right side or left side of head 90 to apply ultrasonic energy to targets on either side of head 90. Three housing arms 112 may extend radially out from sides of outer housing 104. Elevating screws 114 may rotatably extend through housing arms 112 and may include elastomeric cushions 118 that press up against head 90. Elevation screws 114 may be rotated downward pressing against head 90 to reduce some of the compressive force of inner housing 106 against head 90. Alignment system 110 may move the probe within inner housing 106 into different x, y, and/or z positions with respect to head 90. The x position may refer generally to front to back positions with respect to head 90, the y position may refer generally to top to bottom positions of head 90, and the z position may refer generally to a transverse inside to outside, or left to right positions, with respect to head 90. If TNS 100 were attached on the top of head 90, the x position may refer to front to back positions with respect to head 90, the y position may refer to the left to right or side to side positions with respect to head 90, and the z position may refer to the transverse inside or outward or top to bottom positions with respect to head 90. Alignment system 110 may comprise adjustment assemblies 120 and a top adjustment assembly 140 that have the unique ability to move the probe within inner housing 106 in different x, y, and z directions while TNS 100 remains attached to head 90 of patient 88. This allows more precise alignment of the probe with a target location within head 90. Alignment system 110 may also provide quicker and more accurate reattachment of the TNS to head 90 to a same relative position with respect to the target location. This allows TNS 100 to be repeatedly attached during multiple ultrasonic therapy sessions without using an MRI device to relocate the target location. Side adjustment assemblies 120 each include a side adjustment knob 122 that rotatably attaches to a side extension 124 that extends radial out from the side of outer housing 104. Top adjustment assembly 140 includes a top adjustment knob 142 that is rotatably attached to outer housing 104. A threaded ring 146 extends out through the middle of top adjustment knob 142. A top end 144 of a probe lid extends out through threaded ring 146 and a cap 148 inserts into a center cavity of the top end 144 of the probe lid. Cap 148 operates as a wire guide for receiving cable 116 and operates as a stop for top end 144 of the probe lid.

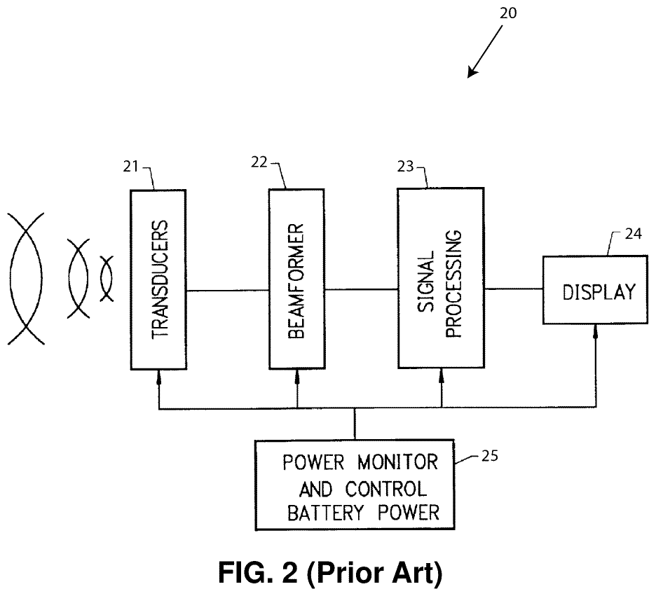

FIG. 2, prior art, is a functional block diagram of a portable ultrasonic diagnostic instrument described in U.S. Pat. No. 6,471,651. Ultrasound probes 21 generate ultrasonic waves and receive reflections of ultrasonic waves. Wave generation and echo signal processing are accomplished by a beamformer circuit 22 which interfaces with the probes 21. Signals from beamformer 22 are passed to a signal processor 23 and the processed signals are used to control a display 24. Electric power for the components of the instrument is provided by a battery source 25 which includes a power monitor and control. Heretofore, portable ultrasonic diagnostic instruments have been available but have operated at power levels exceeding 35 watts. System 20 limits power consumption to 25 watts for a digital beamformer instrument and 10 watts for an analog beamformer instrument.

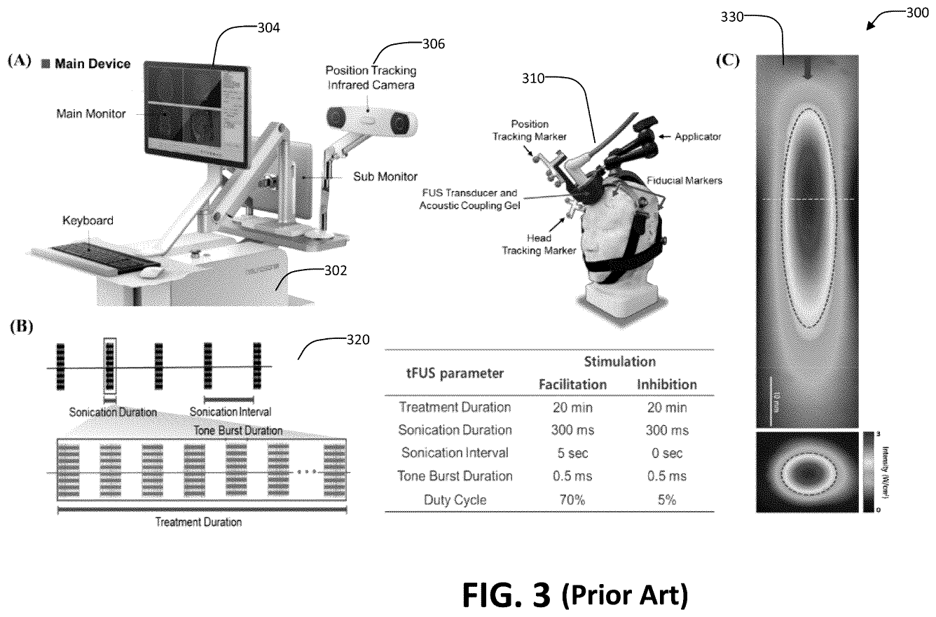

FIG. 3, prior art, illustrates a low intensity tFUS system 300 presented in a paper by Young Goo Kim et al, including a PC 302, a monitor 304, infra-red cameras 306, and an FUS probe 310. FIG. 300 also includes a schematic diagram 320 of sonication parameters (e.g., sonication duration, sonication interval, treatment duration, tone burst duration, duty cycle, etc.) in Group B. The PC 302 controls the operation of the FUS probe 310 to provide stimulation in accordance with the sonication parameters, creating the acoustic profile 330 in lateral and axial planes in Group C. “Stimulation” may more generally be described as “neuromodulation” since ultrasound has the capability to both increase and decrease neural activity, depending on parameters including intensity, burst length, and pulse repetition frequency.

Embodiments of the present disclosure will now be discussed. Methods are described for reliably delivering focused ultrasound to targeted brain anatomy using available ultrasound probes in the form of 1.75 D probe arrays that may be driven by off-the-shelf ultrasound electronic equipment via commercially available cables.

The term “1.75 D” is used in the literature to denote a probe with asymmetric element dimensions. This kind of probe can steer and focus normally in one dimension (which in this patent will be termed “azimuth”) while its capabilities in the other dimension (“elevation”) are reduced because of the small number of elements and their relatively large dimensions in terms of wavelengths. Such probes are the simplest designs that are capable of skull aberration correction and are thus of great importance in FUS systems. They produce slice data in the azimuth plane, and correct aberration in both azimuth and elevation.

While the inability of 1.75 D probes to acquire a volume scan (with high resolution in both azimuth and elevation) is a disadvantage, they are of interest in the development of cost-effective tFUS systems. For example, 1.75 D probes can be manufactured with mature processes using bulk piezoelectric materials by many vendors. Such processes and materials are highly optimized and inexpensive. The interconnect requirements of a 1.75 D probe can be met with off-the-shelf products from vendors such as TE CONNECTIVITY or PROTERIAL CABLE.

An objective of this application is to disclose ways in which 1.75 D probes can form useful parts of a tFUS system. Such systems benefit from low development costs and high component reliability deriving from the use of time-tested products and applications.

Several approaches are disclosed which compensate for the disadvantages of 1.75 D probes for neuromodulation and guidance. In brief, these approaches include:

Starting from an existing, academically developed “average” brain which is adapted to a specific patient's anatomy through the use of optical or acoustic measurements.

Training an AI using a volume-capable matrix probe is a second way to enable the use of 1.75 D probes in clinical practice. After the AI has “seen” many examples of cranial anatomy, it can produce a numerical value corresponding to the accuracy of orientation of a 1.75 D probe as a clinician moves it around on the scalp. A “fuel gauge” display is one useful means to guide the clinician to the correct orientation for a particular patient. This may be coupled with an augmented-reality display in which directions from the AI, such as arrows indicating translation and/or rotation, are superimposed on the live video.

Precise volume data can be acquired even with a 1.75 D probe by using the fact that brain tissue motion within the cranium is insignificant. A mechanical fixture mounted on the patient's head can be used to rotate the probe so that a conical volume is acquired.

These techniques produce partial data about the anatomy of the brain, which is needed to guide the ultrasonic neuromodulation. It is advantageous to estimate the anatomy of the complete brain by using the measured data for content-based image retrieval (CBIR) into an atlas of human anatomical scans (for example, structural MRI scans).

Guidance of the neuromodulation may be achieved using a volumetric 3D database of the head's anatomy. The 1.75 D probe produces only 2D slices of the head. The clinician can move the prove around to get several slices, but that is not as useful as volumetric 3D data such as may be produced by an MRI or a matrix ultrasound array. Since human brains are quite similar n aspects of their anatomies, a database of a few thousand MRIs, for example, can provide data that provides adequate resolution for neuromodulation targeting. Several different features can be collected using a 1.75 D probe that will enable a similarity lookup into a 3D database to find an MRI that closely matches the actual MRI of the subject. Possible features for performing a similarity lookup include:
contours of the inner skull surface opposite the probe positions;
blood vessel data from the 1.75 D probe, as it exhibits a Doppler shift while stationary cranial structures do not;
anatomical features of the head such as ventricles, which are visible under ultrasound and can provide fiducial marks for the similarity lookup.

Once the features are extracted from the ultrasound data, a similarity metric is used to compare the features of the 2D ultrasound data with those in the 3D database. This involves calculating distances between feature vectors. Techniques like k-dimensional trees, locality-sensitive hashing (LSH), and Facebook AI similarity search (FAISS) can be used to index into the MRI database. Once the closest-matching 3D volume has been found, it can be used to guide the neuromodulation.

The slice data produced by a 1.75 D probe may also be placed in the full brain context using an MRI scan of the subject and a NeuroNavigation system, such as the VISOR2 product manufactured by ANT NEURO.

While the 1.75 D array probe is capable only of acquiring a plane of data (typically with a trapezoidal geometry), many types of slice imaging are helpful to guide the neuromodulation beam. These may include gray-scale ultrasound (B-mode), tissue harmonic imaging (THI), Color Doppler and Power Doppler for identifying blood vessels, and strain imaging (acoustic radiation force imaging, ARFI), and shear wave elastography. By avoiding partial delivery of ultrasound energy due to poor guidance or complications due to skull anatomy, unwanted side effects are avoided, and target efficacy is improved.

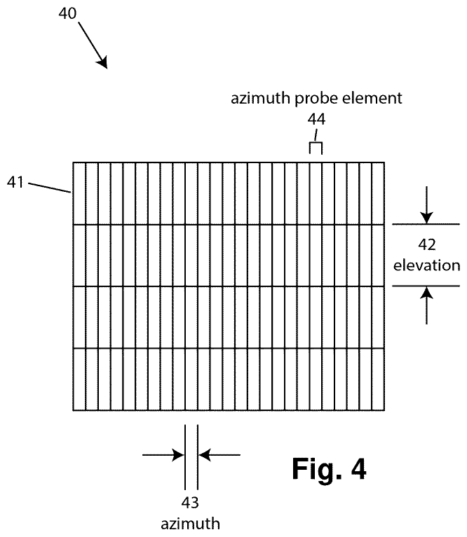

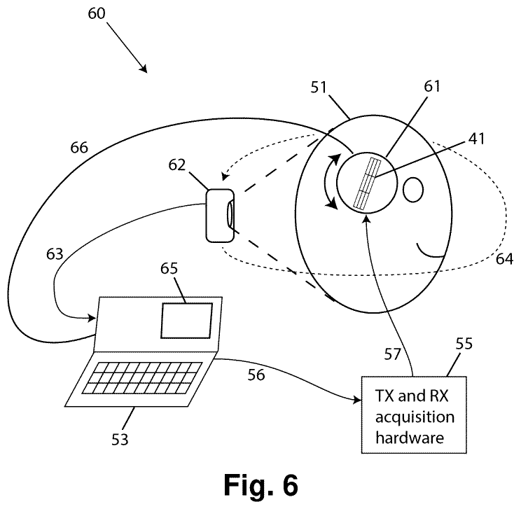

In an embodiment of the present disclosure FIG. 4 illustrates a 1.75 D probe 40 comprising an array 41 of probe elements having elements comprising elevation 42 larger than elements comprising azimuth 43. 1.75 D array 40 is used to steer and focus ultrasonic waves in a tFUS procedure, with variations to be described herein. The azimuth probe element 44 is shown as one column of array 41. The 1.75 D arrangement economizes on the total number of probe elements by emphasizing the more useful azimuth elements over the less useful elevation elements. Efficacy of the accompanying tFUS procedure is achieved with this reduced number of elements, and the cable connecting the probe array 41 to a signal processor becomes implementable as a standard cable, available off-the-shelf. The size of the elements is insufficient to support steering and focusing, but the resolution is sufficient to perform aberration correction in elevation as well as in azimuth. This is because changes in the thickness of the skull are smooth and can be sampled by the larger elevation elements. The data created using probe array 41 may be called “slice data”. To re-phrase the resolution issue, slice data is adequate to perform correct skull aberration, but not adequate to perform steering and focusing without additional functionality, such as a rotatable probe to be discussed in reference to FIG. 6.

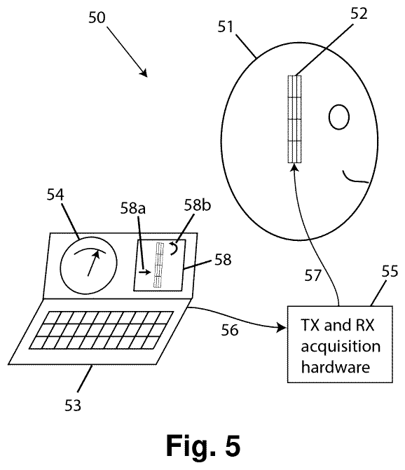

In an embodiment FIG. 5 illustrates a tFUS system 50 wherein a 1.75 D array 52 for acquiring slice data is driven by transmit and receive acquisition hardware 55 which receives input 56 from a computer 53 having a “fuel gauge” display 54. During product development a large amount of volume data from many subjects is acquired using a matrix ultrasound probe. Subsequently, in the clinic, slice data is evaluated by artificial intelligence (AI), performed by an AI-capable processor in computer 53, which has access to the volume data. Computer 53 executes instructions contained in memory and sends control signals 56 to transmit and receive acquisition hardware 55, which controls 57 the probe array 52. The multi-row probe provided in 1.75 D probe array 52 provides adequate resolution to correct skull aberrations, including adaptation to skull thickness. The fuel gauge 54 implements AI in computer 53 to find the optimal slice data for treatment; the gauge peaks when the best plane of acquisition is selected. AI is invoked to evaluate the position and orientation of probe array 52 based on its understanding of human brain anatomy; it indicates through the fuel gauge how suitable the current position and orientation are for guidance of the neuromodulation. The user moves the probe until the reading on the fuel gauge is maximized. User prompts such as arrows 58a and rotation symbols 58b may be provided in a second display 58 to help the operator find the maximum on the fuel gauge.

In an embodiment FIG. 6 illustrates a tFUS system 60 wherein a 1.75 D probe array 41 is used to perform volume guidance. Volume guidance refers to guidance of a neuromodulation beam within a transcranial volume to assist in targeting a treatment zone at a desired site within the volume. A mechanical arrangement 61 allows the rotation of probe 41, which can be motorized or manual. An angular sensor incorporated into mechanical arrangement 61 provides information 66 on the orientation of the probe to computer 53. Mechanical arrangement 61 is used to rotate the 1.75 D probe array 41, while acquiring volume data by moving camera 62 around the subject's head 51 as shown 64. Connection 63 sends video data from camera 62 to computer 53. Computer 53 is connected 56 to transmit and receive acquisition hardware 55 which sends positional commands to mechanical arrangement 61 as shown 57.

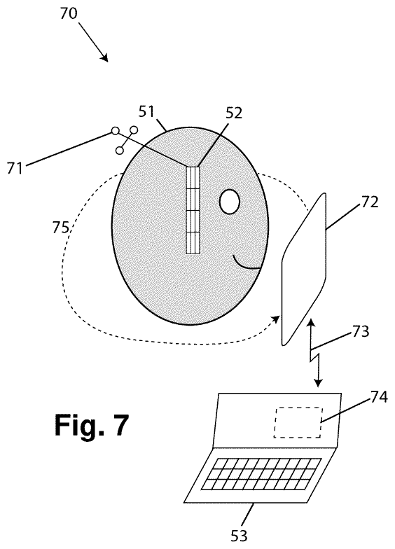

The geometry of the head of subject 51 is acquired optically using a camera 62, informing the deformation of an average head obtained from an MRI database. As described in reference to FIG. 7, LIDAR data can also be used to inform the deformation of an average head. An example of an average head database is the MONTREAL NEUROLOGIC INSTITUTE (MNI) average brain from McGill University. Ultrasound data acquired during the volume scan includes echoes from the inner surface of the far side of the skull from probe 41. The anatomical shape of the skull may be used to further refine the deformed average brain so that it accurately matches the subject anatomy.

Accordingly, this tFUS system 60 can associate head geometry with the location and orientation of the neuromodulation probe, in this case slice data produced by probe array 41. By integrating image data produced by camera 62, content-based image retrieval in a database of MRI volume scans can be employed. Data from this database and optical data produced by camera 62 are displayed on display 65.

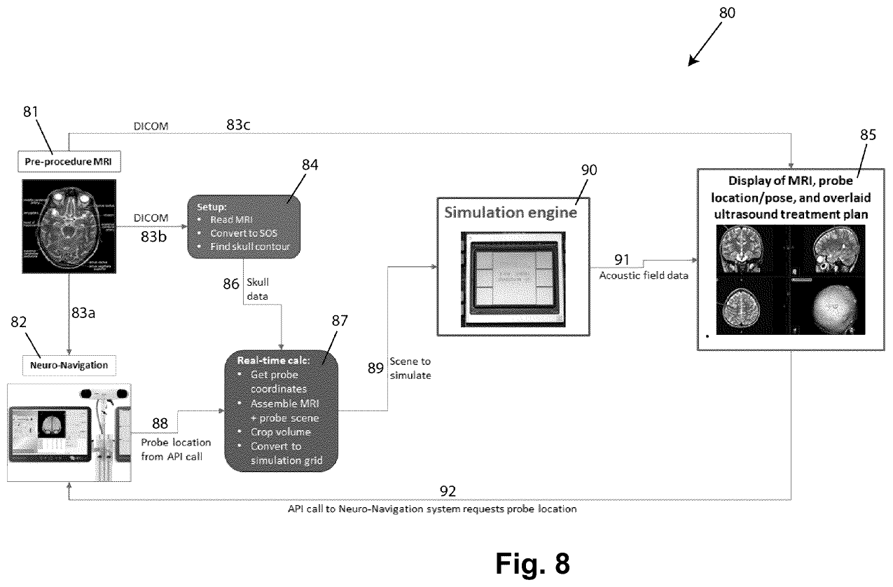

In an embodiment FIG. 7 illustrates a tFUS system 70 having a different form of optically assisted guidance. Subject 51 has a 1.75 D probe array 52 attached as shown, with an optical feature 71 attached to it. A mobile device 72 incorporating a camera is shown. Mobile device 72 is moved around the head 51 of a subject as shown 75; it includes position sensing capabilities such as provided by a Light Detection and Ranging (LIDAR) device. The optical feature 71 is attached to the probe 52, allowing a Neuro Navigation system to report the position and orientation of the probe in the coordinate system of a pre-procedure MRI; this will be further discussed in reference to FIG. 8. Mobile device 72 is linked wirelessly 73 to computer 53 having a display 74. Using bi-directional wireless interface 73 computer 53 can command mobile device 72, and mobile device 72 can report scan data to be displayed by computer 53 using display 74. As previously described in reference to FIG. 6, LIDAR data can also be used to deform the average head.

In an embodiment, FIG. 8 illustrates a tFUS system 80 which can be used to superimpose a simulation of the expected neuromodulation beam pattern on MRI volume data or on ultrasound data. In FIG. 8, pre-procedure MRI data 81 flows in three directions, 83a to a neuro-navigation system 82, 83b to a setup block 84, and 83c to display 85. Links 83b and 83c employ a DICOM interface, an international standard for communicating medical imaging information. Setup block 84 includes the steps of: reading the MRI data; converting to SOS (sinusoidal obstruction syndrome); and determining the skull contour of the subject. Skull data 86 is sent from setup block 84 to a real-time-calculation block 87. As in FIG. 7, the probe is attached to an optical feature recognizable by the Neuro Navigation system. Using a probe location provided 88 by the neuro-navigation system 82, the real-time-calculation block 87 performs the following steps: retrieve probe coordinates; assemble MRI and probe scene; crop the image volume; and convert to a simulation grid. The calculated scene to be used in the simulation is sent 89 to a simulation engine 90 which produces acoustic field data as the simulated input of the expected beam pattern (neuromodulation), and sends it 91 to display 85. Display 85 overlays a display of the MRI volume data, the probe location/pose, and an ultrasound treatment plan. Volume rendering is performed as required to produce the composite images. The computer controlling display 85 makes an API call 92 to the neuro-navigation system to request the probe location. In an alternative embodiment, instead of using a pre-procedure MRI scan, content-based image retrieval may be employed to obtain an approximation of the MRI volume data, as previously described.

System 80 enables a practitioner to see where the ultrasound will focus before starting the neuromodulation; using the mouse to click on the target brain structure ensures that the ultrasound will reach the target. If the simulated data shows that the path to the target is not clinically optimal, the clinician may alter the probe position and repeat the target specification process to access a different path to the target tissue.

What is claimed is:

1. A transcranial ultrasound system comprising:
a probe configured to focus a neuromodulation beam on a patient skull, wherein the probe comprises an array of rectangular elements, wherein a respective elevation of each element of the array is larger than a respective azimuth of each element of the array.

2. The transcranial ultrasound system of claim 1, wherein the probe further comprises a rotatable mechanical structure.

3. The transcranial ultrasound system of claim 1, further comprising:
acquisition hardware coupled to the probe, the acquisition hardware comprising a receiver that receives input from the probe; and
a computing device coupled to the acquisition hardware, the computing device generating a fuel gauge user interface based upon input from the probe, the fuel gauge user interface indicating suitability of a current position or a current orientation of the probe for providing neuromodulation to the patient skull using the probe.

4. The transcranial ultrasound system of claim 3, further comprising:
At least one of a camera or a LIDAR device configured to obtain the current position or the current orientation of the probe relative to the patient skull.

5. The transcranial ultrasound system of claim 4, wherein the at least one of a camera or a LIDAR device is integrated into a mobile device that is separate from the computing device.

6. The transcranial ultrasound system of claim 5, wherein the mobile device is movable about the patient skull to obtain optical feedback associated with the current position or the current orientation.

7. The transcranial ultrasound system of claim 6, wherein the optical feature indicates a position or an orientation of the probe relative to the patient skull.

8. The transcranial ultrasound system of claim 3, wherein guidance information associated with placement of the probe is obtained by the acquisition hardware acoustically measuring an inner surface of the skull on an opposing side of the patient skull from where the probe is positioned.

9. The transcranial ultrasound system of claim 8, wherein the guidance information is received from a pre-procedure MRI and a Neuro-Navigation system.

10. The transcranial ultrasound system of claim 8, wherein the computing device determines the guidance information from an average brain MRI customized to the patient skull using at least one of camera data, LIDAR data, or acoustic data relating to an inner surface of the patient skull.

11. The transcranial ultrasound system of claim 1, wherein the probe further comprises an optical feature comprising visual markings for identification by a camera or a LIDAR device.

12. The transcranial ultrasound system of claim 1, where content-based image retrieval is employed to find a specific brain MRI in a database of brain MRIs, wherein the specific brain MRI is used to provide volume guidance for steering the neuromodulation beam.

13. The transcranial ultrasound system of claim 3, wherein the computing device generates a simulated prediction of an ultrasound neuromodulation beam output by the probe that is overlaid on an image of a brain associated with the patient skull for steering the neuromodulation beam.

14. The transcranial ultrasound system of claim 13, wherein the simulated prediction is derived using a simulator having inputs from a pre-procedure MRI and from a neuro navigation system.

15. The transcranial ultrasound system of claim 14, wherein the pre-procedure MRI is replaced with an approximate 3D MRI obtained using a 1.75 D probe and a similarity lookup into a database of 3D MRI scans.
